(19) United States
(12) Patent Application Publication
(10) Pub. No.: US 2006/0104460 A1
(43) Pub. Date: May 18, 2006

(54) Adaptive time-based noise suppression

(75) Inventors: Ali Behboodian, Natick, MA (US); Marc A. Boillot, Plantation, FL (US); Pratik V. Desai, Boca Raton, FL (US); Chin P. Wong, Coral Springs, FL (US)

Correspondence Address: AKERMAN SENTERFITT, P.O. BOX 3188, WEST PALM BEACH, FL, 33402-3188, US

(73) Assignee: Motorola, Inc., Schaumburg, IL (US)

(21) Appl. No.: 10/992,584

(22) Filed: Nov. 18, 2004

(51) Int. Cl. H03B 29/00; H04B 15/00; A61F 11/06

(52) U.S. Cl. 381/94.1; 381/71.1

(57) ABSTRACT

Systems, an apparatus, and methods are provided for mitigating noise associated with an audio signal. A system (100) for mitigating noise associated with an audio signal includes an estimator module (108). The estimator module determines an estimated level of noise associated with the audio signal. The system also includes an expander module (110). The expander module causes an attenuation of the audio signal if a level of the audio signal is below a signal threshold. The expander module is adaptively tunable so that the attenuation caused (606) by the expander module is based upon the level of noise estimated (602) by the estimator module.

### BACKGROUND

1. Field of the Invention

The present invention is related to the field of electronic communications, and, more particularly, to electronic communications based on audio signals.

2. Description of the Related Art

Noise can degrade the quality of any signal. In the context of electronic communications in which an audio signal is modulated and conveyed via a cellular telephone or other voice-based communication device, noise can distort the signal so that it becomes unintelligible or, at the very least, unpleasant to the listener to whom the communication is directed. A common form of noise that often plagues users of such communication devices is background noise. Background noise includes extraneous speech, termed babble noise, which often permeates a public setting such as a restaurant or other public site. It also includes other extraneous sounds such as music and the like that can interfere with or distort the voice component carried by the audio signal.

Conventional devices have tended to rely on legacy noise suppressors to handle noise. The functional approach of a legacy noise suppressor is typically based on the implementation of a frequency-based algorithm. Although this approach can be successful in reducing white noise, it is not nearly as efficient a technique for dealing with other types of noise, such as that characterized as background noise. This is likely due to the fact that the sort of noise represented by background noise typically shares the same regions of the frequency spectrum of an audio signal as that occupied by speech components of the signal. Legacy noise suppressors, however, are focused mainly on the reduction of white noise that occupies the lower end of the frequency spectrum.

Accordingly, the present art lacks efficient and effective devices or techniques for adequately suppressing noise, especially that characterized as background noise. Moreover, conventional devices and techniques, especially frequency-based ones, largely lack a capability for suppressing noise based upon the estimated level of noise associated with the audio signal. That is, conventional devices and techniques tend not to estimate a level of noise associated with an audio signal and then suppress the audio signal to a greater or lesser extent depending on whether the noise level is estimated to be relatively high or relatively low.

### SUMMARY OF THE INVENTION

One aspect of the present invention is an adaptive, time-based system for mitigating noise associated with an audio signal. The system can include an estimator module, the estimator module determining an estimated level of noise associated with the audio signal. The system additionally can include an expander module for causing an attenuation of the audio signal if a level of the audio signal is below a signal threshold. The expander module can be adaptively tunable in the sense that the attenuation of the audio signal caused by the expander module can be based upon the level of noise estimated by the estimator module. According to one embodiment, for relatively high estimated levels of noise, the expander module can cause a relatively high degree of attenuation of the underlying audio signal. Conversely, for relatively low estimated levels of noise, the expander module can cause a relatively low degree of attenuation, according to this embodiment.

Another aspect of the present invention is a method for mitigating noise associated with an audio signal. The method can include determining an estimated level of noise associated with the audio signal, and causing an attenuation of the audio signal if a level of the audio signal is below a signal threshold. The attenuation of the audio signal can be based upon the estimated level of noise. More particularly, according to another embodiment, the attenuation can be greater the greater the estimated level of noise.

Yet another aspect of the present invention is an apparatus comprising a computer-readable storage medium. The storage medium can include computer instructions for mitigating noise associated with an audio signal. The computer instructions can include instructions for determining an estimated level of noise associated with the audio signal. The computer instructions further can include instructions for causing an attenuation of the audio signal if a level of the audio signal is below a signal threshold, the attenuation of the audio signal being based upon the estimated level of noise.

### BRIEF DESCRIPTION OF THE DRAWINGS

There are shown in the drawings, several embodiments, it being understood, however, that the invention is not limited to the precise arrangements and instrumentalities shown.

FIG. 1 is a schematic diagram of a communication device that includes a system for mitigating noise associated with an audio signal, according to an embodiment of the present invention.

FIG. 2 is a detailed schematic diagram of the system illustrated in FIG. 1

FIG. 3 illustrates an expansion curve based upon an audio signal attenuated according to an embodiment of the present invention.

FIGS. 4a-4c illustrate expansion curves based upon an audio signal attenuated according to yet another embodiment of the present invention.

FIG. 5 is a curve showing the functional relationship between a beta parameter and an estimated level of noise associated with an audio signal according to still another embodiment of the present invention.

FIG. 6 is a flowchart of a method of mitigating noise associated with an audio signal, according to yet an additional embodiment of the present invention.

### DETAILED DESCRIPTION OF THE INVENTION

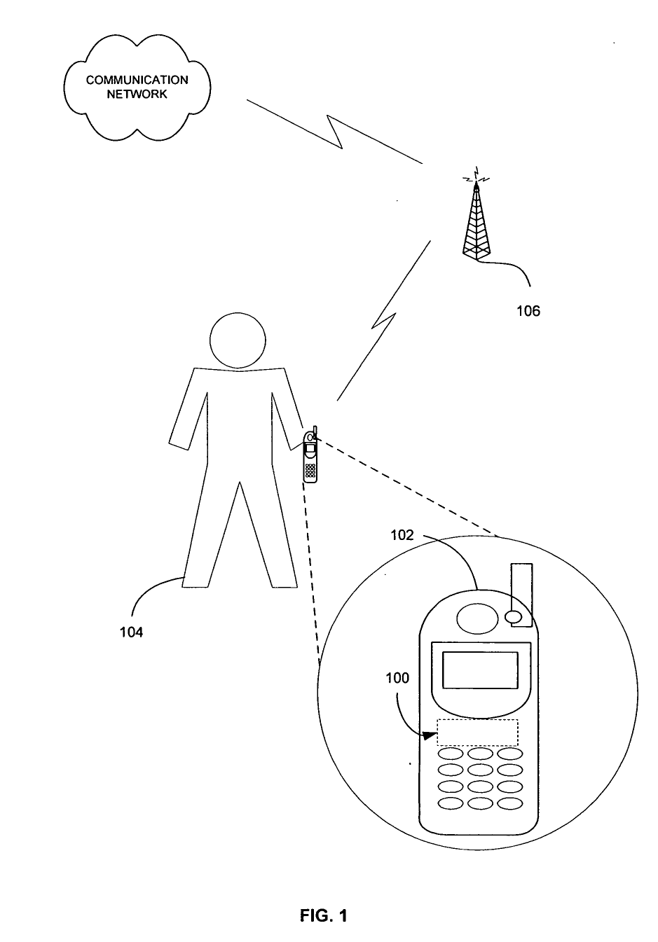

FIG. 1 is a schematic diagram of a system 100 for mitigating noise associated with an audio signal in accordance with an embodiment of the inventive arrangements disclosed herein. As illustrated, the system 100 can be included in a communication device 102, such as a cellular telephone, for improving communications conducted by an individual 104 using the device to communicate, via a remote site 106, with a communication network. It will be readily apparent from the ensuing discussion that the system 100 alternately can be integrated in, connected to, or otherwise communicatively linked with various other types of communication and electronic devices that convey, process, or similarly utilize audio signals, as described herein.

The audio signal can comprise any modulated electrical signal that becomes sound when amplified and converted to acoustic vibrations by an audio output device such as a speaker (not shown). More particularly, the audio signal can be an electrical signal associated with a communication device 102 such as the Integrated Digital Enhanced Network (iDEN) device by Motorola, Incorporated, of Schaumburg, Ill. The communication device 102 alternately can be any other type of electronic device by which various modes of communication are effected through the use of audio signals, the audio signals being in the form of input and/or output comprising modulated electrical signals that are processed to produce sound.

The noise associated with the audio signal can comprise any extraneous signal component which tends to interfere with or disturb the sound or quality of the signal present in or passing through the communication device 102. In the context of a communication device, for example, noise can comprise background noise such as music or so-called babble noise, such as the extraneous speech that permeates a public setting such as a restaurant or other public site.

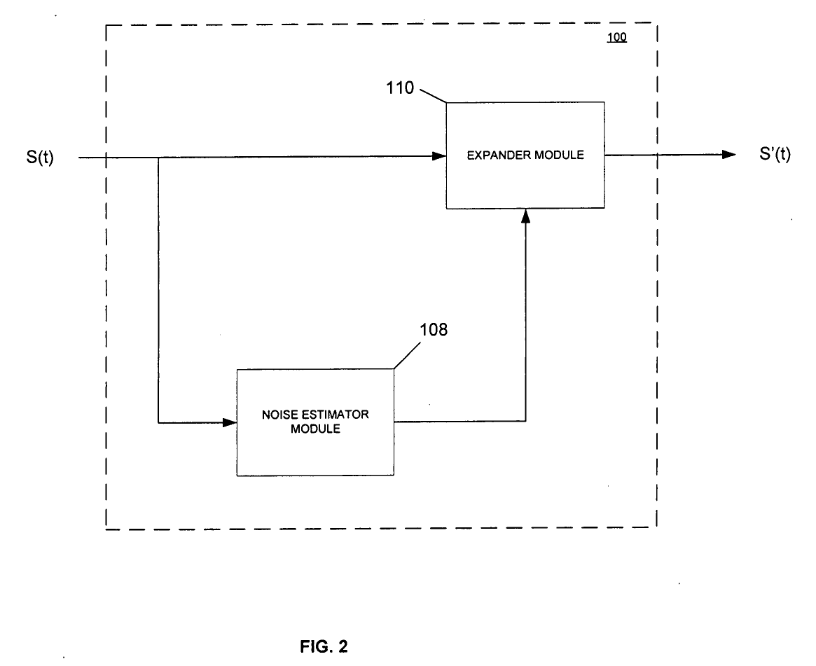

Referring additionally to FIG. 2, the system 100 illustratively includes an estimator module 108 for determining an estimated level of noise associated with the audio signal. The system 100 also illustratively includes an expander module 110 for causing an attenuation of the audio signal if a level of the audio signal is below a signal threshold. The expander module 110 is adaptively tunable in the sense that the attenuation of the audio signal is based upon the estimated level of noise.

The adaptive tuning, according to one embodiment described in more detail below, enables the system 100 to attenuate or suppress the audio signal less when there is less noise associated with the audio signal, and to attenuate or suppress more when there is more noise associated with the audio signal. According to another embodiment also described in more detail below, the threshold is adjusted based upon the estimated level of noise. Accordingly, the threshold is set so as to be more stringent, the greater the noise level is estimated to be, and to be less stringent the less the noise is estimated to be.

The estimator module 108 estimates the level of noise associated with the audio signal illustratively received by the communication device 102. The level of noise can be estimated, according to one embodiment, by analyzing multi-sample speech frames. A multi-sample speech frame, as will be readily understood by one of ordinary skill in the art, can be generated by the communication device 102 using a speech encoder (not shown). The speech encoder samples the audio signal and uses the samples to generate encoded data that represents the audio signal. The encoded data, in turn, is aggregated to form distinct, multi-sample speech frames.

For example, variable-rate speech encoders are now commonly used in wireless communication devices because they can increase the lifespan of batteries used to power the devices and because they increase system capacity with relatively slight impact on perceived speech quality. The Telecommunications Industry Association has codified the most popular variable rate speech encoders standards such as the Interim Standard IS-96 and Interim Standard IS-733. These variable rate speech encoders encode the speech signal at four possible rates referred to as full rate, half rate, quarter rate or eighth rate according to the level of voice activity, the rate corresponding to the number of bits used to encode a frame of speech. The rate can vary on a frame-by-frame basis. For many such communication devices a speech frame can comprise 180 samples per frame.

In accordance with one embodiment, the estimator module 108 estimates the level of noise by computing an average, or mean, of absolute values of the signal level of each of the samples that comprise a multi-sample frame. A signal level, as will be readily understood by one of ordinary skill in the art, corresponds to the energy content of a signal. In the current context, the signal level illustratively corresponds to the energy associated with each sample of the multi-sample frame. For a 180-sample speech frame, therefore, the level of noise, as estimated by the estimator module 108, can be based on a sum of 180 absolute signal level values, the sum being divided by 180.

According to still another embodiment, the estimated noise level is updated by the estimator module 108 on an on-going, dynamic basis. The dynamically estimated level of noise can be defined, for example, by the following equation:
EBNᵢ=EBNᵢ₋₁+(1−β)*AVSF,

where EBNᵢ denotes the current estimated level of noise associated with the audio signal received by the communication device 102, EBNᵢ₋₁ denotes a previous estimated level of noise, AVSF denotes the absolute value of a current speech frame, and β denotes a parameter representing a rate at which the estimated level of noise is dynamically estimated.

The key parameter in the equation EBNᵢ=EBNᵢ₋₁+(1−β)*AVSF is β. The parameter β determines the rate at which the current estimate of the level of noise, EBNᵢ is updated or revised. A value for β can be calculated by comparing the absolute value of the current speech frame, AVSF, with the estimated level of noise, as determined by the difference equation EBNᵢ=EBNᵢ₋₁+(1−β)*AVSF. Whether and to what extend β is updated depends on which of three distinct conditions obtained during audio signal processing by the communication device 102 exists.

First, if the absolute value of the current speech frame is at least equal to some multiple (greater than one) times the estimated level of noise, then it can be assumed that the frame, or, more precisely the portion of the audio signal represented by the frame, contains more than mere noise—it contains actual speech. In this case, β is set equal to one. Consistent with the assumption that the underlying audio signal contains actual speech, an efficient approach is to set the greater-than-one multiple equal to 2, such that the estimated level of noise is multiplied by 2. Thus, β will be set to one whenever AVSF>2*EBNᵢ.

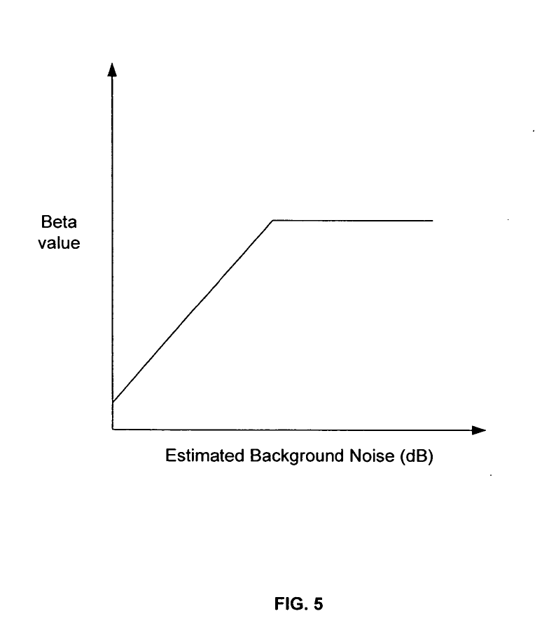

Conversely, if the absolute value of the current speech frame is less than the estimated level of noise, then β is revised or updated. This follows since it can be assumed that the underlying signal, if it contains more than mere noise, will be at least equal to the estimated level of noise. In this case, β can be set to a predetermined value reflecting the rate at which it is desired to update the parameter. In the third and final case, if the absolute value of the speech frame lies between the estimated level of noise and some multiple greater than one (e.g. 2) times the estimated level of noise, then β can be updated according to the following equation:
β=max[clip(2*EBNᵢ)−param₁,param₂],

where, again, param₁ and param₂ can be chosen based upon a desired rate for updating the parameter β. The equation as given ensures that β is less than a maximum (by virtue of the inclusion of param₁) and yet remains greater than zero (if β becomes zero, the updating process stops). In general, the equation ensures that rate at which β is updated varies inversely with the estimated level of noise; a high level of noise induces slower updating of β and vice versa. FIG. 5 is a graph representing one of the different functional relationships that can exist between the parameter β and the level of noise estimated by the estimator module 108 in accordance with the mathematical form described above.

The expander module 110 causes a downward expansion of the underlying audio signal if the level of the audio signal falls below a threshold. In general, the threshold is set at a level below a desired level, but above a noise floor. When the audio signal drops below the threshold, the expander module 110 causes an attenuation or further reduction in the audio signal. Since it is reasonable to assume that the drop in the signal level is indicative of a lack of voice content, the suppression of the below-threshold signal is intended to reduce the remaining signal component, the noise. Thus, the signal threshold is set such that it is below some minimum desired level, the threshold, but above the noise “floor.” When the audio signal drops below the threshold, the expander module 110 suppresses or attenuates the audio signal so that its signal level drops even further. According to one embodiment, the amount of signal suppression or attenuation is a function of the estimated level of noise, as determined by the estimator module 108. That is, the level of noise estimated by the estimator module 108 determines the extent to which the expander module 110 suppresses or attenuated the underlying audio signal.

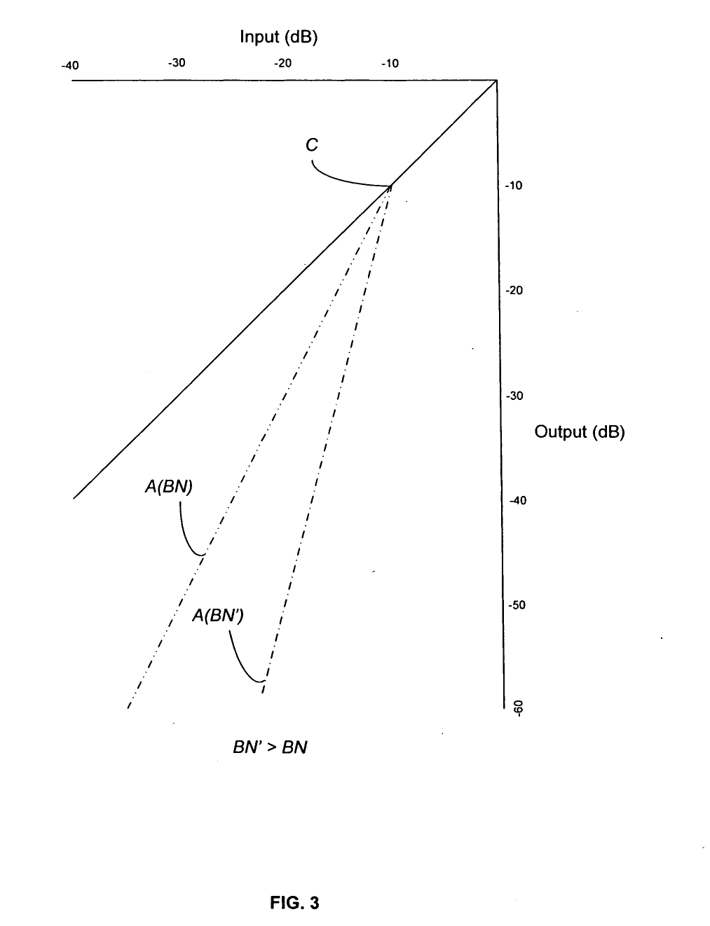

FIG. 3 illustrates the embodiment according to which the attenuation caused by the expander module 110 is greater or less depending on whether the noise associated with the audio signal is greater or less. As illustrated by the curve A(BN), there is a threshold, denoted by the corner point C, below which the expander module 110 causes an attenuation of the audio signal. The threshold is illustratively at a signal level of −10 decibels (dB). Beyond that point, a −10 dB change in the level of the audio signal (i.e., the input) results in an attenuated signal (i.e., the output) that is attenuated by −2 dB for each one dB drop in the audio signal. The rate of attenuation is based on the estimated noise level, BN, associated with the audio signal.

In accordance with this embodiment, the rate of attenuation is greater if the estimated noise level associated with the audio signal is BN′, where BN′>BN, as shown in FIG. 3. In this case, as illustrated by the curve A(BN′), the expander module causes an attenuation of −4 dB for each one dB drop in the audio signal once the audio signal drops to a level below the −10 dB threshold (corner point C). Thus, according to this embodiment, an increase in the noise level, BN, estimated by the estimator module 108, results in the adaptively tunable expander module 110 causing a greater attenuation of the audio signal.

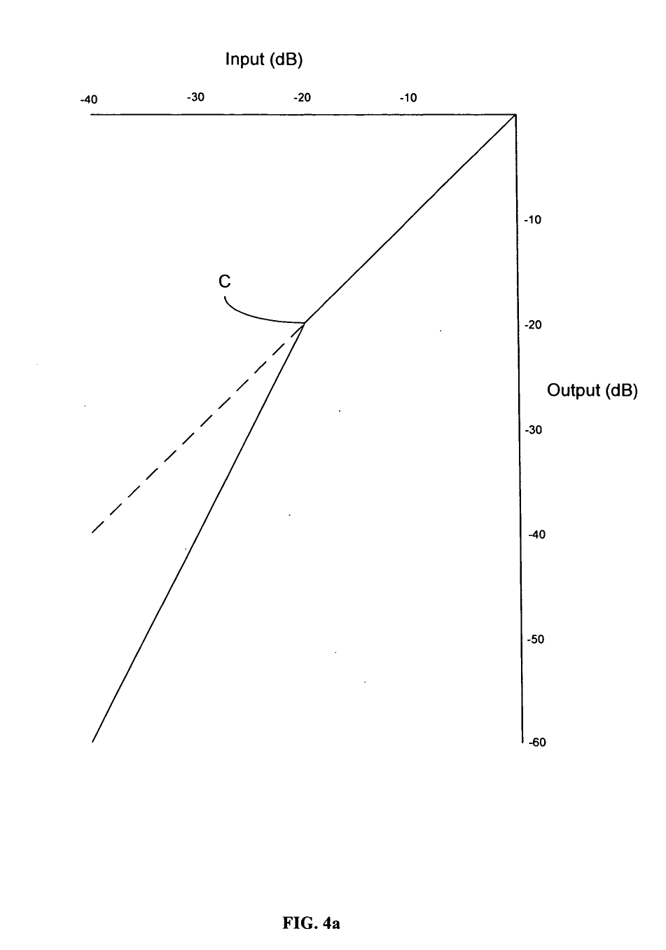

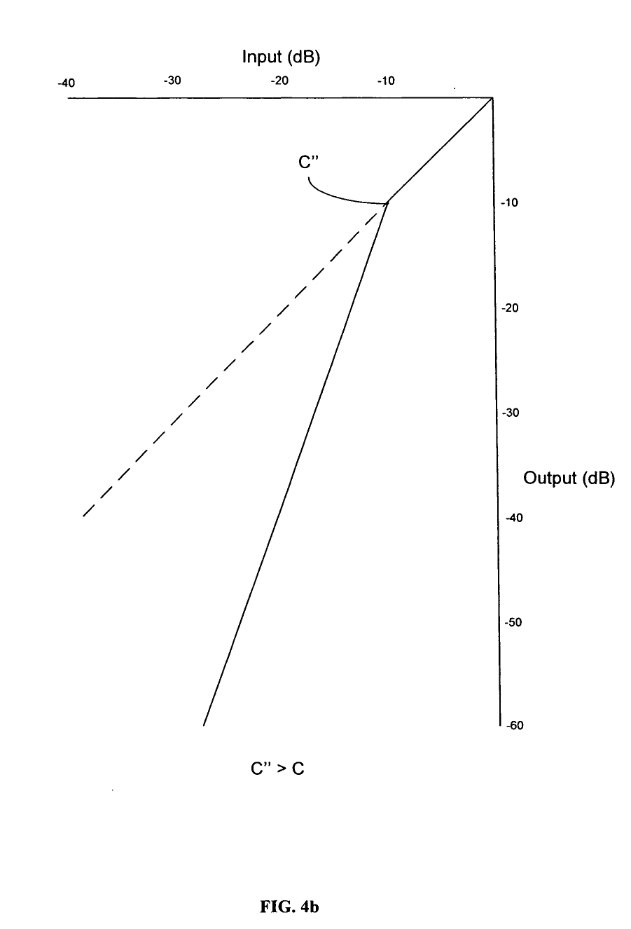

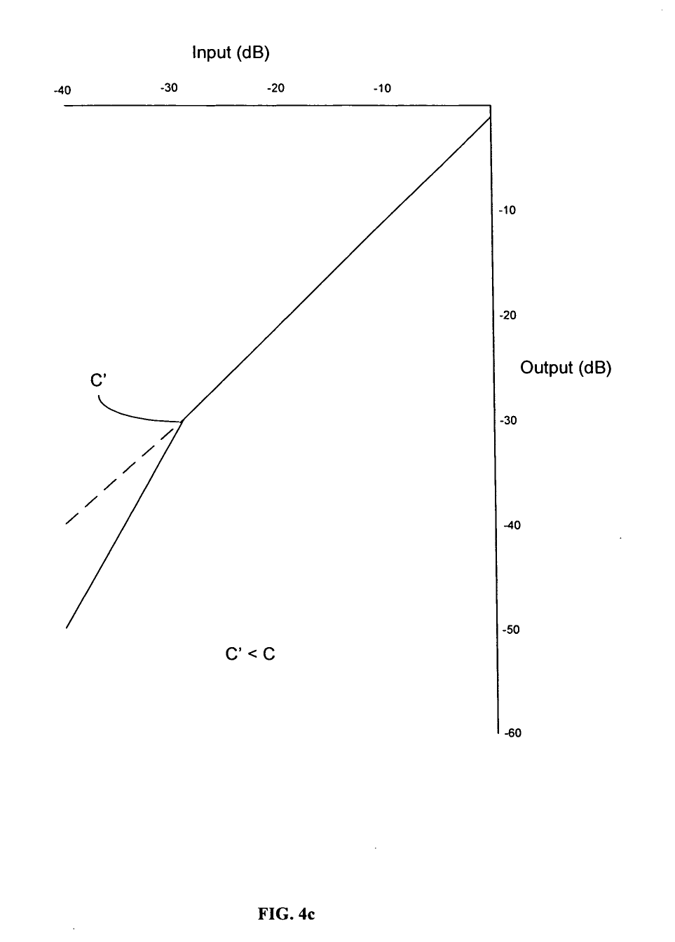

According to yet another embodiment, as illustrated by the different expansion curves in FIG. 4a-FIG. 4c, the expander module 110 causes an attenuation of the audio signal by establishing the signal threshold based upon the level of noise estimated by the estimator module 108. FIG. 4a illustratively provides a benchmark in which the threshold is set at a signal level of −20 dB, as shown by the corner point C. If the estimated level of noise associated with the audio signal increases, then the expander module 110 illustratively sets the threshold at −10 dB, as shown by the corner point C′ in FIG. 4b. Conversely, as illustrated by FIG. 4c, if the estimated noise level is relatively lower, then the expander module 110 accordingly sets the threshold at −30 dB, as shown by corner point C″.

Note that with respect to each of the expansion curves, FIG. 4a-FIG. 4c, the threshold represented by the corner point depends on the estimated level of noise. More particularly, the higher the level of noise, the more stringent the threshold set by the expander module 110; that is, for a moderate level of noise, a −20 dB drop in the audio signal is needed to induce the expander. For a relatively high level of noise, the drop need be only −10 dB, whereas for a relatively low level of noise, a drop of −30 dB or more is need before the expander module 110 causes further attenuation of the audio signal.

The signal threshold as determined by the expander module 110 can be defined by a mathematical relationship based upon the estimated level of noise determined by the estimator module 108. For example, the signal threshold can be defined by the following the linear relationship in which C, again, denotes the corner point, BN denotes the estimated level of noise, and S denotes a shift parameter:
C=BN+S.

The exemplary expansion curves, above, can be mathematically described by the following equation, in which y denotes the attenuated audio signal (i.e., output), x denotes the audio signal (i.e., input), a denotes slope of the portion of the curve corresponding to an input signal level below the threshold, and C is defined as above:
y=αx−C(α−1).

Accordingly, the amount of attenuation caused by the expander module 110 based upon the estimated level of noise determined by the estimator module 108 can be expressed by the following equation, wherein the amount of attenuation corresponds to the difference between the attenuated audio signal (output) and the audio signal (input), denoted by A:
Δ=y−x=(α−1)(x−C).
Substituting BN−S for C in the previous equation yields:
Δ=(α−1)(x−BN−S).

It is worth noting that if the audio signal comprises only noise, such as background noise, then the absence of audio-based input results in the last equation being reduced to the following formulation:
Δ=−(α−1)S.

The amount of gain associated with the signal can also be calculated. For a time index, i, the gain is G(i). Recalling that, in general, a scaling factor in the dB domain equates to a compression in the linear (time) domain, it follows that a*X(t), in the dB domain, equates to x(t)^{a}, in the linear (time) domain. From above, in the dB domain, Δ=(α−1)(x−c). Accordingly, the gain can be derived as follows:
G(i)=10^{Δ/10}=10^{(α-1)(x-c)/10}
10^{[x(α-1)+c(1-α)]}=10^{x(α-1)/10}10^{c(1-α)/10}=C_{log}^{(1-α)}·|x(i)|^{(α-1)}
Considering that for |x(i)|>C_{log} the gain is one, we can have a general equation for the gain as follows:
G(i)=C_{log}^{(1-α)}min(C_{log},|x(i)|)^{(α-1)}, where C_{log}=10^{c/10}

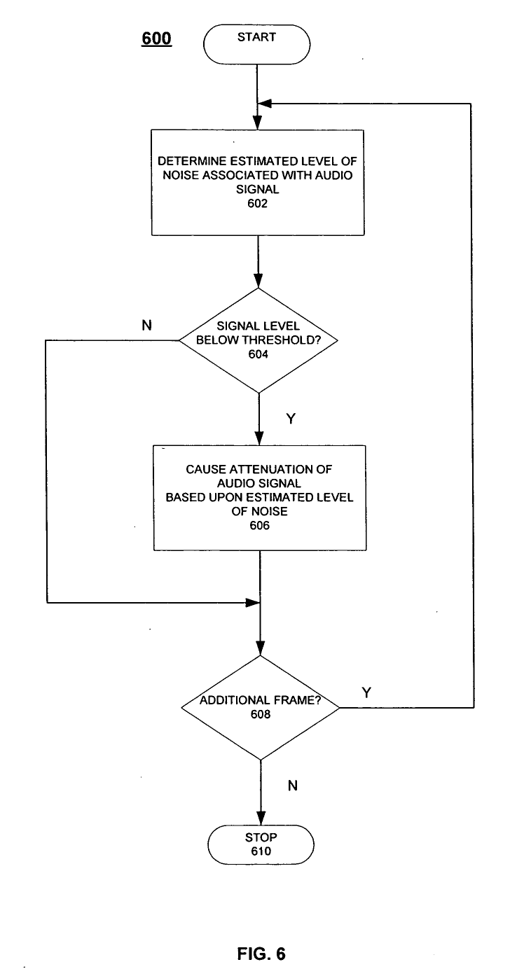

FIG. 6 is a flowchart of a method 600 for mitigating noise associated with an audio signal, according to still another embodiment. The method includes, at step 602, determining an estimated level of noise associated with the audio signal. At step 604, a determination is made as to whether the audio signal is below a predetermined threshold. If so, an attenuation of the audio signal is caused at step 606, wherein the attenuation is based upon the level of noise estimated at step 602. The method 600 is illustratively applied with respect to an audio signal that is represented by multi-sample speech frames. Accordingly, each of the steps can be applied on a frame-by-frame basis. Therefore, at step 608 a determination is made as to whether there is a multi-sample frame to which the method thus far has not been applied. If so, the method 600 continues by returning to step 602 and repeating the remaining steps. The steps are repeated until the method 600 has been applied to each multi-sample frame corresponding to the particular audio signal being processed at which point the method 600 can stop at step 610.

The present invention can be realized in hardware, software, or a combination of hardware and software. The present invention can be realized in a centralized fashion in one computer system, or in a distributed fashion where different elements are spread across several interconnected computer systems. Any kind of computer system or other apparatus adapted for carrying out the methods described herein is suited. A typical combination of hardware and software can be a general purpose computer system with a computer program that, when being loaded and executed, controls the computer system such that it carries out the methods described herein.

The present invention also can be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

This invention can be embodied in other forms without departing from the spirit or essential attributes thereof. Accordingly, reference should be made to the following claims, rather than to the foregoing specification, as indicating the scope of the invention.

What is claimed is:

1. An adaptive, time-based system for mitigating noise associated with an audio signal, the system comprising:
an estimator module for determining an estimated level of noise associated with the audio signal; and
an expander module for causing an attenuation of the audio signal if a level of the audio signal is below a signal threshold, said expander module being adaptively tunable so that the attenuation of the audio signal is based upon the estimated level of noise.

2. The system of claim 1, wherein the expander module causes an attenuation based upon the estimated level of noise by setting the signal threshold based upon the estimated level of noise.

3. The system of claim 2, wherein the signal threshold is linearly related to the estimated level of noise.

4. The system of claim 3, wherein the signal threshold is defined by C=BN+S, C denoting the signal threshold, BN denoting the estimated level of noise, and S denoting a shift parameter.

5. The system of claim 4, wherein the attenuation of the audio signal is defined by Δ=(α−1)(x−C)=(α−1)(x−BN−S), Δ denoting the attenuation, x denoting the level of the audio signal, a denoting a quantitative relationship between the level of the audio signal and an output based upon the audio signal, BN denoting the estimated level of noise, and S denoting the shift parameter.

6. The system of claim 1, wherein the estimated level of noise is dynamically estimated based upon a prior estimated level of noise and an average value corresponding to a current speech frame derived from the audio signal.

7. The system of claim 6, wherein the dynamically estimated level of noise is defined by EBNᵢ=EBNᵢ₋₁+(1−β)*AVSF, EBNᵢ denoting a current estimated level of noise, EBNᵢ₋₁ denoting a previous estimated level of noise, AVSF denoting the average value corresponding to a current speech frame, and β denoting a parameter representing a rate at which the estimated level of noise is dynamically estimated.

8. A time-based method for adaptively mitigating noise associated with an audio signal, the method comprising:
determining an estimated level of noise associated with the audio signal; and
causing an attenuation of the audio signal if a level of the audio signal is below a signal threshold, the attenuation of the audio signal being based upon the estimated level of noise.

9. The method of claim 8, wherein causing the attenuation comprises determining the signal threshold based upon the estimated level of noise.

10. The method of claim 9, wherein the signal threshold is determined based upon a linear relationship between the signal threshold and the estimated level of noise.

11. The method of claim 10, wherein the linear relationship is defined by C=BN+S, C denoting the signal threshold, BN denoting the estimated level of noise, and S denoting a shift parameter.

12. The method of claim 11, wherein the audio signal is attenuated based upon a linear relationship between the attenuation and both the estimated level of noise and the shift parameter, the relationship defined by Δ=(α−1)(x−C)=(α−1)(x−BN−S), Δ denoting the attenuation, x denoting the level of the audio signal, a denoting a quantitative relationship between the level of the audio signal and an output based upon the audio signal, BN denoting the estimated level of noise, and S denoting the shift parameter.

13. The method of claim 8, further comprising dynamically estimating a current level of noise based upon a prior estimated level of noise and an average value corresponding to a current speech frame derived from the audio signal.

14. The method of claim 13, dynamically estimating the current level of noise comprises ed level of noise is defined by EBNᵢ=EBNᵢ₋₁+(1−β)*AVSF, EBNᵢ denoting a current estimated level of noise, EBNᵢ₋₁ denoting a previous estimated level of noise, AVSF denoting the average value corresponding to a current speech frame, and β denoting a parameter representing a rate at which the estimated level of noise is dynamically estimated.

15. A computer-readable storage medium, the storage medium comprising computer instructions for:
determining an estimated level of noise associated with the audio signal; and
causing an attenuation of the audio signal if a level of the audio signal is below a signal threshold, the attenuation of the audio signal being based upon the estimated level of noise.

16. The computer-readable storage medium of claim 15, further comprising a computer instruction for determining the signal threshold based upon the estimated level of noise.

17. The computer-readable storage medium of claim 16, wherein the signal threshold is determined based upon a linear relationship between the signal threshold and the estimated level of noise.

18. The computer-readable storage medium of claim 17, wherein the linear relationship is defined by C=BN+S, C denoting the signal threshold, BN denoting the estimated level of noise, and S denoting a shift parameter.

19. The computer-readable storage medium of claim 15, further a computer instruction for dynamically estimating a current level of noise based upon a prior estimated level of noise and an absolute value of a current speech frame.

20. The computer-readable storage medium of claim 19, wherein dynamically estimating the current level of noise comprises ed level of noise is defined by EBNᵢ=EBNᵢ₋₁+(1−β)*AVSF, EBNᵢ denoting a current estimated level of noise, EBNᵢ₋₁ denoting a previous estimated level of noise, AVSF denoting the average value corresponding to a current speech frame, and β denoting a parameter representing a rate at which the estimated level of noise is dynamically estimated.
